(19) United States
(12) Patent Application Publication
(10) Pub. No.: US 2015/0305788 A1
(43) Pub. Date: Oct. 29, 2015

(54) PROSTHESIS MEMBER OF A JOINT PROSTHESIS FOR A THUMB JOINT AND JOINT PROSTHESIS

(71) Applicant: SWEMAC INNOVATION AB, Lidköping (SE)

(72) Inventors: Henrik HANSSON, Vreta Kloster (SE); Lars ÖSTER, Lidköping (SE)

(21) Appl. No.: 14/441,863

(22) PCT Filed: Nov. 16, 2012
(86) PCT No.: PCT/SE2012/051269

§ 371 Date: May 11, 2015

(51) Int. Cl. A61B 17/86

(52) U.S. Cl. CPC A61B 17/8685

(57) ABSTRACT

A prosthesis member of a joint prosthesis for a thumb joint comprises a screw-like part (4) and an articulating socket element (6) with a mounting pin (9) which is insertable into a hole (8) in the screw-like part for attaching the articulating socket element thereto. The screw-like part (4) of the prosthesis member (2) is configured with a first fixing portion (15) which is provided with a first external thread (17) for attachment by screwing and fastening of said prosthesis member to the hard outer part (T1) of the trapezium (T), and a second fixing portion (18) having a less diameter than the diameter of the first fixing portion and being provided with a second external thread (19) which is configured to permit attachment by screwing and fastening of said prosthesis member to the soft inner part (T2) of the trapezium. The first fixing portion (15) is provided with a surface (20) which connects the threads (17, 19) on said fixing portions (15, 18) to each other and which converges substantially arcuate towards the second fixing portion (18). Hereby, optimum fixation of the prosthesis member (2) in the trapezium (T) is achieved.

### TECHNICAL FIELD

The present invention relates to a prosthesis member of a joint prosthesis for a thumb joint, wherein the prosthesis member comprises a screw-like part which is adapted for being attached by screwing into the trapezium at the thumb joint and an articulating socket element, and wherein the articulating socket element has a mounting pin which is insertable into a hole in the screw-like part for attaching the articulating socket element thereto. The present invention also relates to a joint prosthesis for a thumb joint, wherein the joint prosthesis comprises a prosthesis member as defined above and a prosthesis member comprising a screw-like part which is adapted for being attached by screwing into the first metacarpal bone at the thumb joint and an articulating ball element.

### BACKGROUND OF THE INVENTION

The joint prosthesis at which the prosthesis member according to the invention is adapted for use at first hand, comprises two prosthesis members of which one is adapted for attachment to the trapezium and the other to the first metacarpal bone at a thumb joint. The prosthesis member for the trapezium comprises a first screw-like part and the prosthesis member for the first metacarpal bone a second screw-like part. These screw-like parts are adapted for being attached by screwing into the respective bone. The prosthesis member for the trapezium also comprises an articulated socket element and the prosthesis member for the first metacarpal bone an articulated ball element. The articulated socket element has a mounting pin for insertion into a hole in the first screw-like part for attaching the articulating socket element thereto and the articulated ball element has a mounting pin for insertion into a hole in the second screw-like part for attaching the articulating ball element thereto.

A joint prosthesis for a thumb joint can be attached with as well as without bone cement. For attachment without bone cement, problems that the prosthesis loosen often arise. This is true particularly on the trapezium side. There is also a risk that the prosthesis member for the trapezium “settles”, i.e. that it sinks ulnarly and gets a totally different angle with the result that the prosthesis is subluxated (dislocated).

A joint prosthesis of the above-mentioned type wherein particularly the prosthesis member with the articulating socket element, i.e. the prosthesis member for the trapezium, now has been given a new configuration in accordance with the present invention, is described in e.g. SE 533 438 C2. This joint prosthesis operates substantially satisfactory, but is despite its construction not quite optimal.

### SUMMARY OF THE INVENTION

Therefore, the object of the present invention is primarily to eliminate the problem that prosthesis members for the trapezium loosen or “settle”. This is arrived at by providing such a prosthesis member with the characterizing features of subsequent claim 1.

Since the screw-like part of the prosthesis member with the articulating socket element is configured with a first fixing portion, defining an opening into the hole as well as portions of the hole for the mounting pin of the articulating socket element close to the opening and provided with a first external thread which is configured to permit attachment by screwing and fastening of the said prosthesis member to the hard outer part of the trapezium, and a second fixing portion having a less diameter than the diameter of the first fixing portion, defining a bottom to the hole as well as portions of the hole for the mounting pin of the articulating socket element close to the bottom and provided with a second external thread which is configured to permit attachment by screwing and fastening of said prosthesis member to the soft inner part of the trapezium, and since said first fixing portion is provided with a surface without threads, said surface connecting the threads on said fixing portions to each other and converging substantially arcuate towards the second fixing portion, optimum fixation of the prosthesis member in the trapezium is achieved and the prosthesis member is effectively prevented from sinking into the trapezium.

By means of the relatively wider thread on the first fixing portion, maximum fixation in the cortical bone of the trapezium is achieved. By means of the wider first fixing portion and the wider thread, the moments generated during loading is minimized because said moments can be spread over a relative large surface. This also gives place to the articulating socket element. The center of movement of the ball in the socket is located down in the bone, in vertical direction as close as possible to the thread for the cortical bone in the trapezium, and not over the bone. By means of the relatively seen less wide thread in the second fixing portion, efficient fixation in the spongious bone of the trapezium is achieved without fissuring or crack formation.

A joint prosthesis comprising a prosthesis member of improved construction and function as defined above, result in that the entire joint prosthesis will work better.

The object of the present invention is also to provide a prosthesis member which is configured such that the risk for luxation is reduced and, if luxation should occur, which permits simple and quick restoration of the joint.

By the unique configuration according to the invention of the screw-like part, an operator can choose if he or she will use an articulating socket element of e.g. a plastic or metallic material.

### BRIEF DESCRIPTION OF THE DRAWINGS

The above-mentioned and other objects of the invention, the characterizing features thereof and the advantages achieved thereby will be further described in the following with reference to the accompanying drawings, in which

FIG. 1 illustrates with a side view a first embodiment of a thumb joint prosthesis according to the present invention prior to implantation thereof in the hand;

FIG. 2A-2C illustrate with a side view, a perspective view and a plan view a first screw-like part of the prosthesis member for the trapezium of the thumb joint prosthesis according to FIG. 1 prior to location thereof in the trapezium;

FIG. 2D illustrates with a schematic perspective view the first screw-like part of the prosthesis member for the trapezium after location thereof in the trapezium;

FIG. 3 illustrates with a schematic sectional view a somewhat modified first screw-like part of the prosthesis member for the trapezium after location thereof in the trapezium;

FIGS. 4A and 4B illustrate with a perspective view and a side view an articulating socket element, forming part of the thumb joint prosthesis according to FIG. 1, for attachment thereof in the first screw-like part according to FIG. 2A-2C or FIG. 3 of the prosthesis member for the trapezium;

FIG. 5 illustrates with a schematic sectional view the first screw-like part according to FIG. 2A-2D and the articulating socket element fitting thereto according to FIGS. 4A and 4B of the prosthesis member for the trapezium after assembly thereof;

FIG. 6 illustrates with side view corresponding to FIG. 1 a second embodiment of the thumb joint prosthesis according to the present invention with, included therein, an alternatively configured articulating socket element for the first screw-like part according to FIG. 2A-2D and FIG. 3 of the prosthesis member for the trapezium;

FIG. 7 illustrates with a perspective view an instrument for forced driving into the trapezium of the first screw-like part of the prosthesis member for the trapezium; and

FIG. 8 illustrates with a schematic sectional view the driving instrument in operating position in a screw-like part.

### DETAILED DESCRIPTION OF PREFERRED EMBODIMENTS

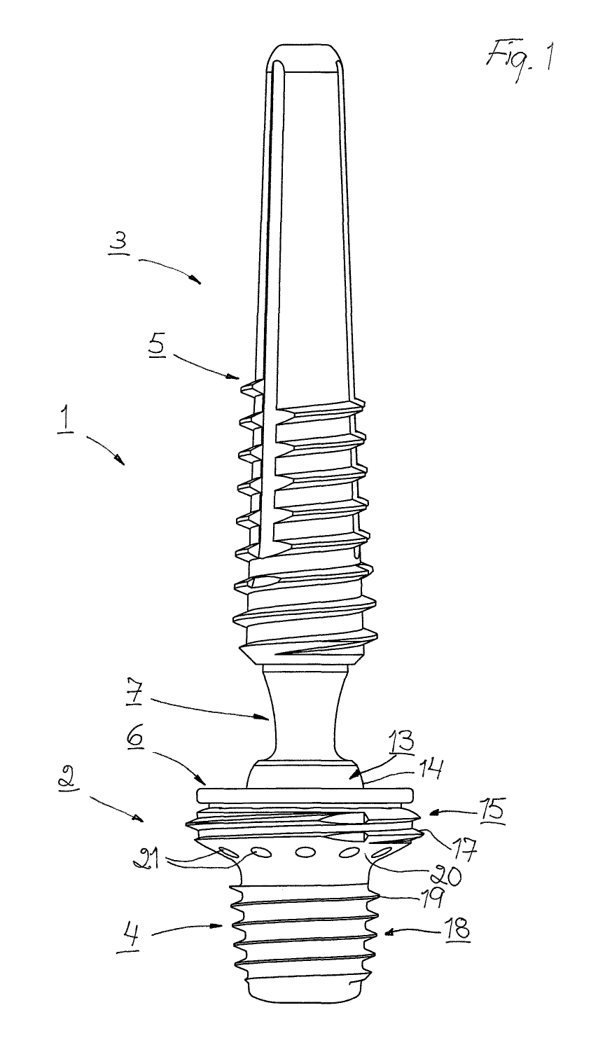

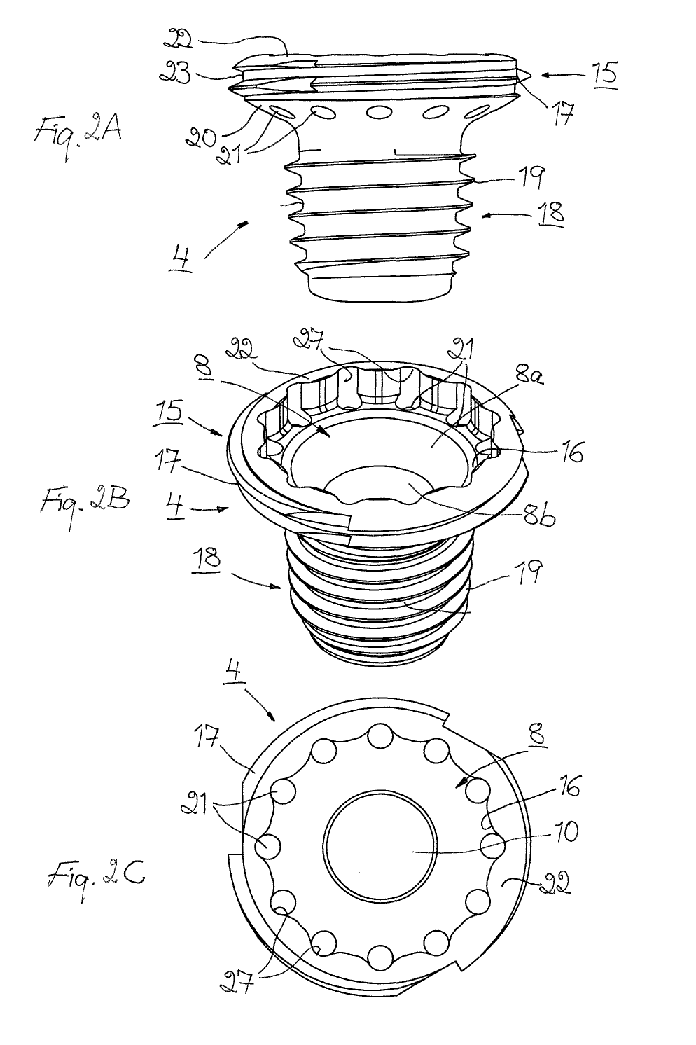

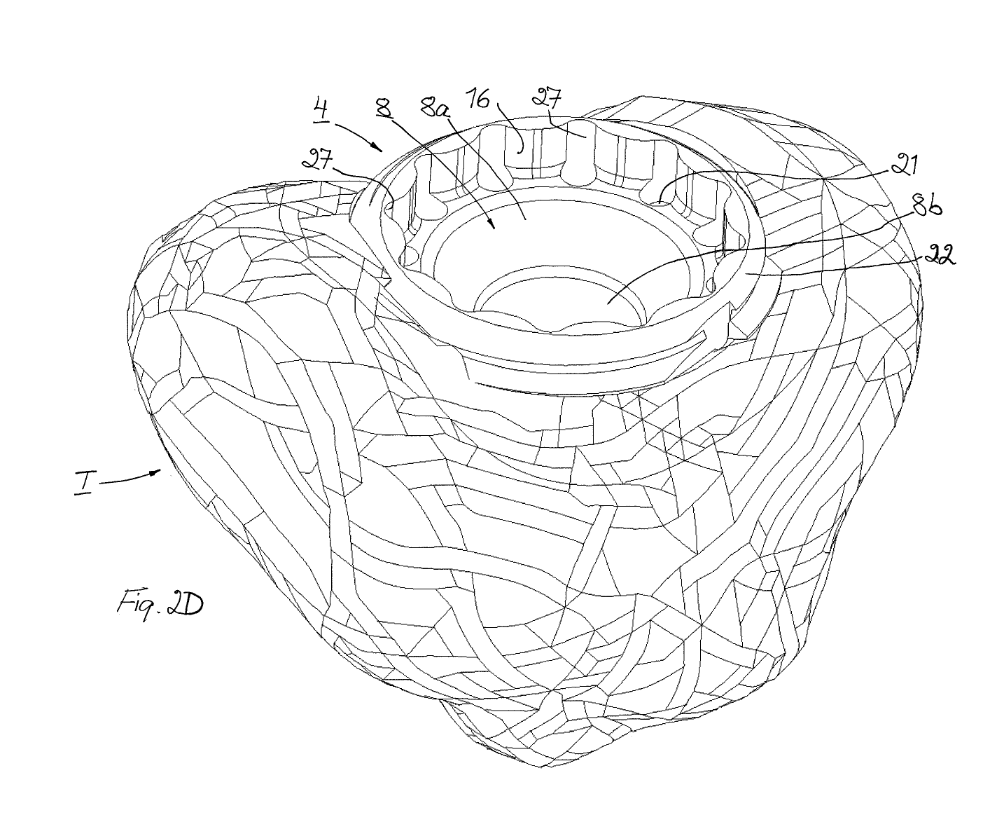

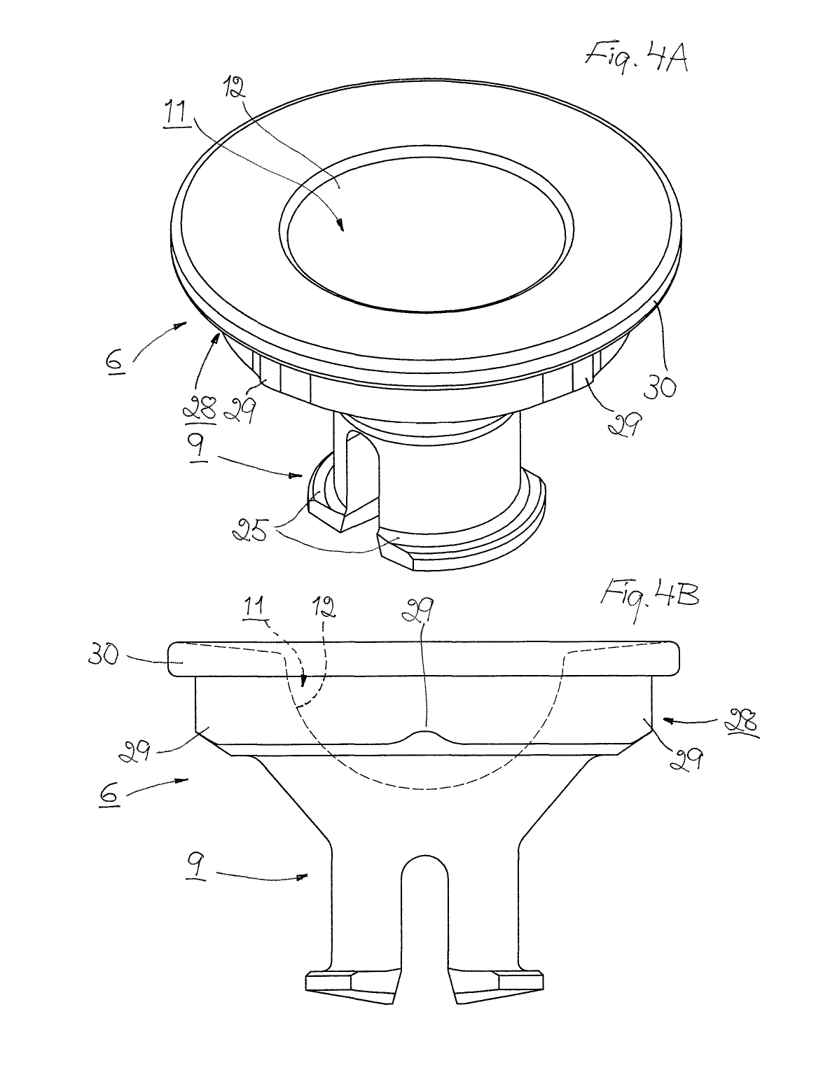

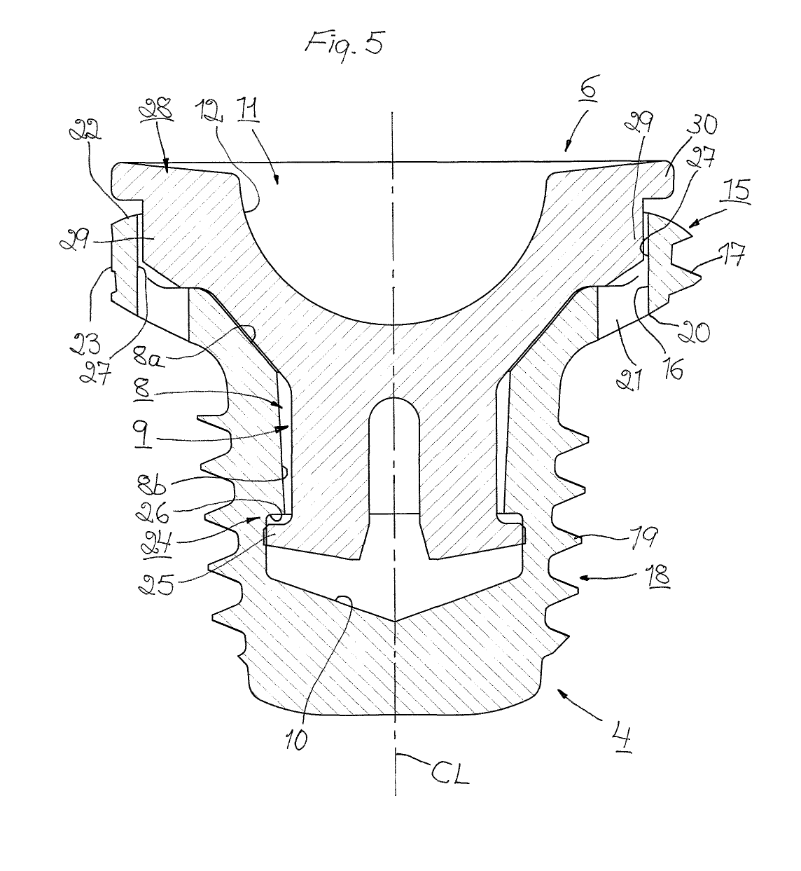

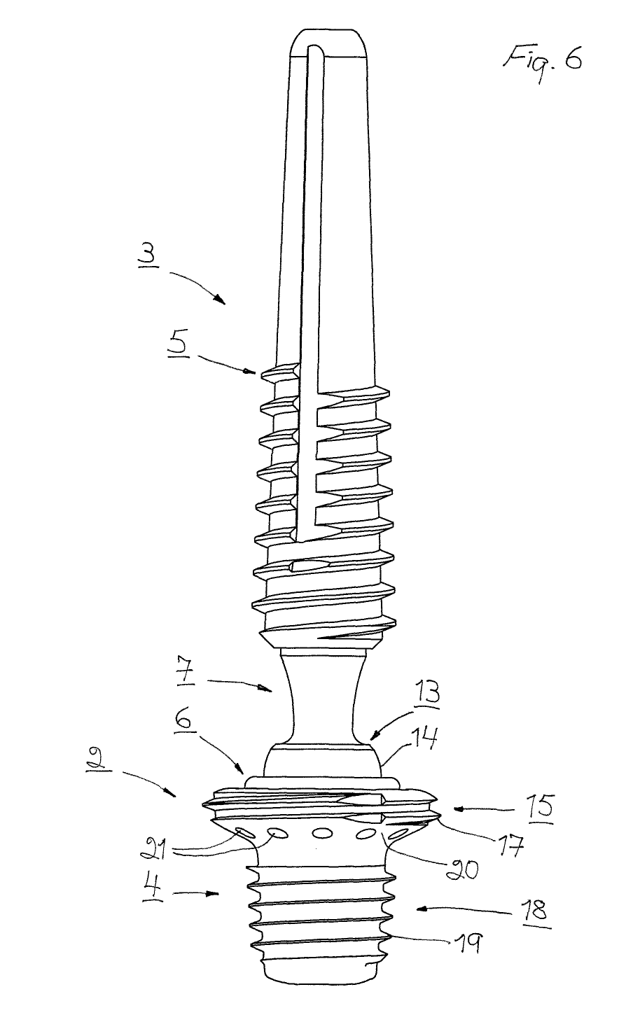

In drawing FIGS. 1 and 6 there is illustrated a first and second preferred embodiment respectively, of a thumb joint prosthesis 1. The thumb joint prosthesis 1 comprises a first prosthesis member 2 and a second prosthesis member 3. The first prosthesis member 2 comprises a first screw-like part 4 which is adapted for being attached by screwing into the trapezium T at the thumb joint and the second prosthesis member 3 comprises a second screw-like part 5 which is adapted for being attached by screwing into the first metacarpal bone (not illustrated) at the thumb joint. The screw-like parts 4, 5 are for this purpose configured with threads which entirely or partly extend in the longitudinal direction thereof. The first prosthesis member 2 also comprises an articulating socket element 6 and the second prosthesis member 3 comprises an articulating ball element 7. The first and second screw-like parts 4, 5 respectively, are in the illustrated embodiments provided with an attachment portion which consists of a hole for the articulating socket and ball elements 6, 7 respectively. In the drawings, only the hole 8 for the articulating socket element 6 in the first screw-like part 4 (see FIGS. 2B, 2C, 2D and 5) is shown. The articulating socket and ball elements 6, 7 respectively, are in turn each configured with a mounting pin which is insertable into the hole in the first and second screw-like parts 4, 5 respectively, for location of the articulating socket and ball elements in a respective screw-like part. In the drawings, only the mounting pin 9 on the articulating socket element 6 is shown (see FIGS. 4A, 4B and 5).

The hole 8 in the first screw-like part 4 extends into said part 4 as a depression and is in the illustrated embodiment centered with a geometric centre line CL extending axially through the part 4. The hole 8 in the first screw-like part 4 has in the illustrated embodiment a shape which is tapering conically towards the bottom 10 of said hole. The conicity may be unchanged in the longitudinal direction of the hole or, as in the illustrated embodiment, may vary in the longitudinal direction of the hole.

The hole in the second screw-like part 5 is preferably provided and configured conically in the same way as the hole 8 in the first screw-like part 4, but may also be configured in any other suitable way.

The articulating socket element 6 comprises, beyond the mounting pin 9, a socket 11 which defines a concave joint or guide surface 12. The mounting pin 9 projects in an axial direction from the outer side of the socket 11. The mounting pin 9 may taper conically from the socket 11. The shape and size of the mounting pin 9 and the shape and size of the hole 8 in the first screw-like part 4 may be selected such that they form a press fit by moving them together in an axial direction, i.e. such a connection which permits bringing the articulating socket element 6 and the first screw-like part 4 to attach to each other by pressing them together. The shape and size of the mounting pin 9 on the articulating socket element 6 and the shape and size of the hole 8 in the first screw-like part 4 may also be selected such that they attach to each other in another way, which is described in more detail below.

The articulating ball element 7 comprises a substantially spherical ball 13 which defines a convex joint or guide surface 14 of such shape that it fits in the guide surface 12 of the socket 11 such that said guide surfaces 12, 14 can slide against each other and provide articulation of the joint. The mounting pin on the articulating ball element 7 extends in an axial direction from the ball 13 and this mounting pin has preferably a corresponding conical shape as the mounting pin 9 on the articulating socket element 6, but may also be configured in any other suitable manner. The shape and size of the mounting pin on the articulating ball element 7 and the shape and size of the hole in the second screw-like part 5 may be selected such that they by moving them together in an axial direction can form a press fit corresponding to the press fit between the articulating socket element 6 and the first screw-like part 4. The shape and size of the mounting pin on the articulating ball element 7 and the shape and size of the hole in the second screw-like part 5 may also be selected such that they attach to each other in another way. It is also possible to make the second screw-like part 5 and the articulating ball element 7 in one piece.

The (first) screw-like part 4 of the (first) prosthesis member 2 comprises according to the invention a first fixing portion 15 which defines an opening 16 into the hole 8 and portions 8a of the hole close to the opening. The first fixing portion 15 is in turn provided with a first external thread 17 which is configured to permit attachment by screwing and fastening or fixation of said prosthesis member 2 in the hard outer part T1 of the trapezium T, i.e. in the cortical bone therein. The screw-like part 4 further comprises a second fixing portion 18 which defines the bottom 10 of the hole 8 and portions 8b of the hole close to the bottom. The second fixing portion 18 has a smaller diameter than the first fixing portion 15. The second fixing portion 18 is also provided with a (second) external thread 19 which is configured to permit attachment by screwing and fastening of the prosthesis member 2 in the soft inner part T2 of the trapezium T, i.e. in the spongious bone therein. The threads 17, 19 on the fixing portions 15, 18 may extend along the entire respective fixing portion or along portions thereof. Exteriorly, each fixing portion 15, 18 has in the illustrated embodiment a constantly tapering conical shape. With a correctly adapted, not too aggressive conical shape, fissuring or crack formation is further reduced in the various parts of the trapezium.

As already indicated above, the relative wider thread 17 on the first fixing portion 15 provides for maximum fixation in the cortical bone T1 in the trapezium T. During uneven weight or load distribution, the thread 17 retains the screw-like part 4 in fixed position in the trapezium T. By means of the wider first fixing portion 15 and the wider thread 17, the moments generated during loading are kept at a minimum, since these moments can be spread over a relative large surface. This also provides for much room in particularly the opening 16 of the hole 8 for the socket 11 in the articulating socket element 6, e.g. room for a socket having a diameter of about 6 millimeters. Rotation of the prosthesis member 2 is prevented. The center of movement for the ball 13 in the socket 11 is found down in the bone, vertically as close as possible to the thread for the cortical bone T1 in the trapezium T and not one or more millimeters above said bone. Thereby, the risk for luxation is also reduced. By means of the relatively seen less wider thread 19 in the second fixing portion 18, efficient fixation in the spongious bone T2 in the trapezium T is achieved without fissuring or crack formation therein.

The length of the first and second fixing portions 15, 18 is optimally adapted to the thickness of the cortical bone T1 in the trapezium T and to the thickness of the spongious bone T2 in the trapezium respectively. The total length of the screw-like part 4 may be e.g. 7, 8.5 or 10 millimeters, but other lengths are of course also possible. Important is that the size of the screw-like part 4 is such that it in position does not penetrate the trapezium T at any articulating surface, that e.g. the length of the screw-like part is such that the bottom thereof is located so close to the articulating surface of scaphoideum or scaphoid bone (not shown), maybe 2 to 3 millimeters, that bone growth around the screw-like part is promoted.

According to the invention, the first fixing portion 15 of the (first) screw-like part 4 of the (first) prosthesis member 2 also comprises a surface 20 which connects the threads 17, 19 on the fixing portions 15, 18 to each other and which converges substantially arcuate towards the second fixing portion 18. This surface 20 has no thread. By means of the arcuately converging configuration of the surface 20, the distribution or spreading of the load or weight moments is further facilitated and reduces the shearing forces on the prosthesis member 2.

The external threads 17, 19 on the first and second fixing portions 15, 18 have in the illustrated embodiment the same pitch, e.g. a pitch of about one millimeter, and are conical, i.e. follow the shape of the fixing portions. This in order to avoid fissuring or crack formation in the various parts of the trapezium T. The threads 17, 19 however, may alternatively be straight and, if desired, may have different pitches. In order to provide for an optimum grip and to simplify and shorten the time for attaching by screwing the screw-like part 4 into the trapezium T, the external threads 17, 19 may be self-tapping.

The first fixing portion 15 of the (first) screw-like part 4 of the (first) prosthesis member 2 is in the illustrated embodiment configured substantially as a screw head-like portion with through holes 21 which are configured to permit ingrowth of bone tissue therein. These holes 21 open into the opening 16 to the hole 8 for the mounting pin 9 of the articulating socket element 6 as well as into the arcuately converging surface 20 between the first and second fixing portions 15, 18. In the illustrated embodiments, there are twelve such holes 21, uniformly distributed peripherally around the hole 8, but the number may of course vary. The first fixing portion 15 also has, after attachment by screwing into the trapezium T, a proximal side 22 which defines the opening 16. This side 22 is substantially saddle-shaped in order to adapt as far as possible to the shape of the articulating surface on the trapezium T which is facing the first metacarpal bone and which said side 22 is substantially in level with. The first fixing portion 15 also includes a lateral surface 23 with the external thread 17 and, after attachment by screwing into the trapezium T, a distal side defining the surface 20 interconnecting the threads 17, 19 of the fixing portions 15, 18. The second fixing portion 18 of the (first) screw-like part 4 of the (first) prosthesis member 2 is in the illustrated embodiments configured substantially as a screw shaft-like portion.

The hole 8 in the (first) screw-like part 4 and the mounting pin 9 of the articulating socket element 6 are configured with complementary portions for fastening or fixation of the articulating socket element in the screw-like part.

Depending on particularly the type of articulating socket element 6, these portions may, as indicated above, be configured to form a press fit such that the articulating socket element and the first screw-like part 4 by means of a press fit are brought to attach to each other. This is true particularly if both members 4, 6 are made of metal or a metal alloy, e.g. if e.g. the articulating socket element 6 is made of a CoCrMo-alloy. At the embodiment of the thumb joint prosthesis according to FIG. 6, the articulating socket element 6 is made of a CoCrMo-alloy.

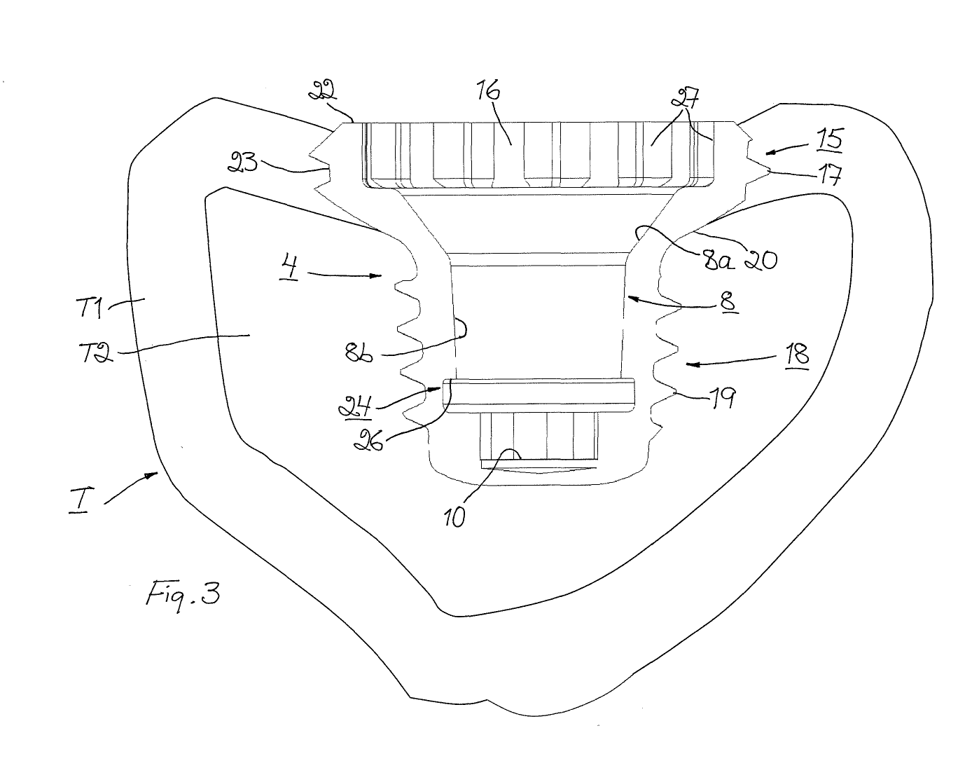

However, if the articulating socket element 6 consists of e.g. a plastic material, e.g. PEEK (polyetheretherketon), which might be reinforced with carbon fibers, the complementary attachment portions may be configured differently, as indicated above. An articulating socket element of PEEK is illustrated in FIGS. 1, 4A, 4B and 5. At this latter embodiment, the hole 8 in the screw-like part 4 may at the bottom 10 be provided with a snap-in attachment 24 for the mounting pin 9 of the articulating socket element 6 and the mounting pin is configured with snap-in portions 25 which engage the snap-in attachment in the hole in said screw-like part. As is apparent from FIGS. 3 and 5, the snap-in attachment 24 in the hole 8 is defined by a smaller extension of the hole in the parts 8b thereof close to the bottom 10 of the hole, and the snap-in portions 25 on the mounting pin 9 are configured as hook-like parts. From FIGS. 4A, 4B and 5 it is apparent that the mounting pin 9, in order to permit a snap-in connection thereof in the hole 8, is divided into two members with each a hook-like part. The mounting pin 9 may of course also be divided into more than two members and each such member may in turn have more than one hook-like part. The snap-in portions 25 configured as hooks on the mounting pin 9 of the articulating socket element 6, are brought together during insertion of the mounting pin in the hole 8, but move apart in the extended part of the hole and prevent retraction of the articulating socket element by engaging the surface 26 defined by said extension and facing the bottom 10 of the hole.

The hole 8 in the (first) screw-like part 4 and a portion 28 of the articulating socket element 6 defining the socket 11, are also configured with complementary portions which prevent rotation of the articulating socket element relative to the screw-like part.

At the embodiment of FIGS. 2B, 2C, 2D, 3 and 5, the hole 8 in the screw-like part 4 is to this end on the inside of the opening 16 thereto provided with at least one recess 27 and the articulating socket member 6 has according to FIGS. 4A, 4B and 5 externally on the portion 28 defining the socket 11 at least one protrusion 29 which engage the recess in the screw-like part. As is apparent from FIGS. 2B, 2C, 2D and 5, said at least one recess on the inside of the opening 16 to the hole 8 may be configured as a number of recesses 27 uniformly distributed along said inside and substantially coinciding with the holes 21 in the first fixing portion 15. The side of the recesses 27 has the same or substantially the same radius as the holes 21. The number of recesses 27 may vary and be more or less than twelve. From FIGS. 4A, 4B and 5 it is apparent that said at least one protrusion on the portion 28 may comprise four protrusions 29 uniformly distributed peripherally on the outside of said portion and having a curved side with substantially the same radius as the recesses 27. The number of protrusions 29 may also vary and be more or less than four, but not more than the number of recesses 27.

The articulating socket element 6 may comprise, except for the mounting pin 9 and except for the portion 28 defining the socket 11 and having at least one protrusion 29, a collar 30 on said portion. The object of this collar 30, which protrudes a little bit more over the screw-like part 4 than said metallic articulating socket element, is, inter alia, to permit restoration without metalosis at a possible luxation. Since the articulating socket element 6 of metal is a bit lower, restoration at luxation is still easier than at older constructions.

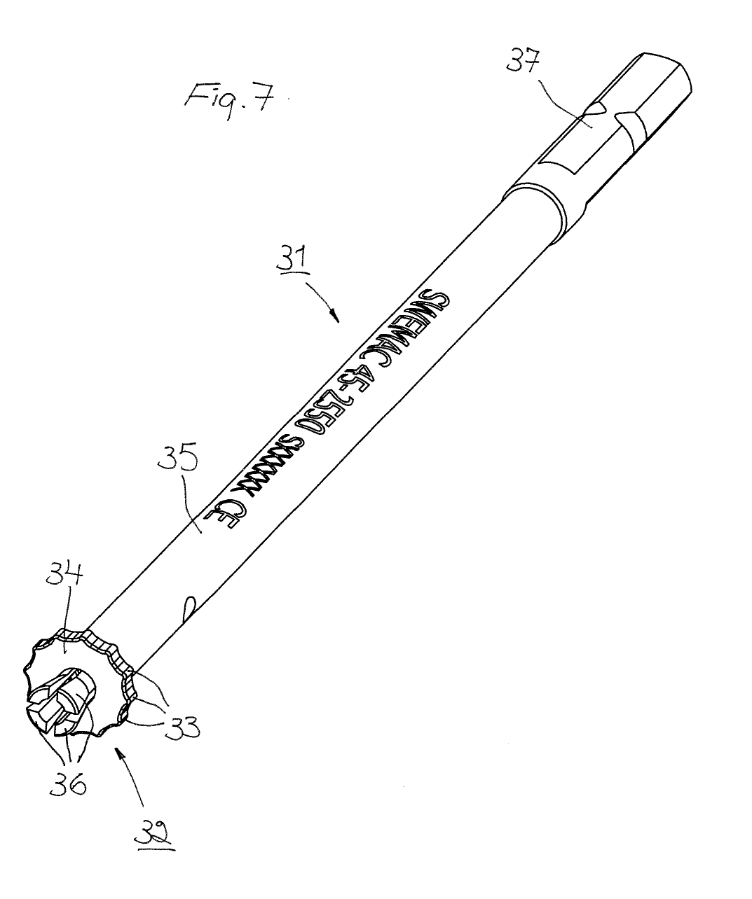

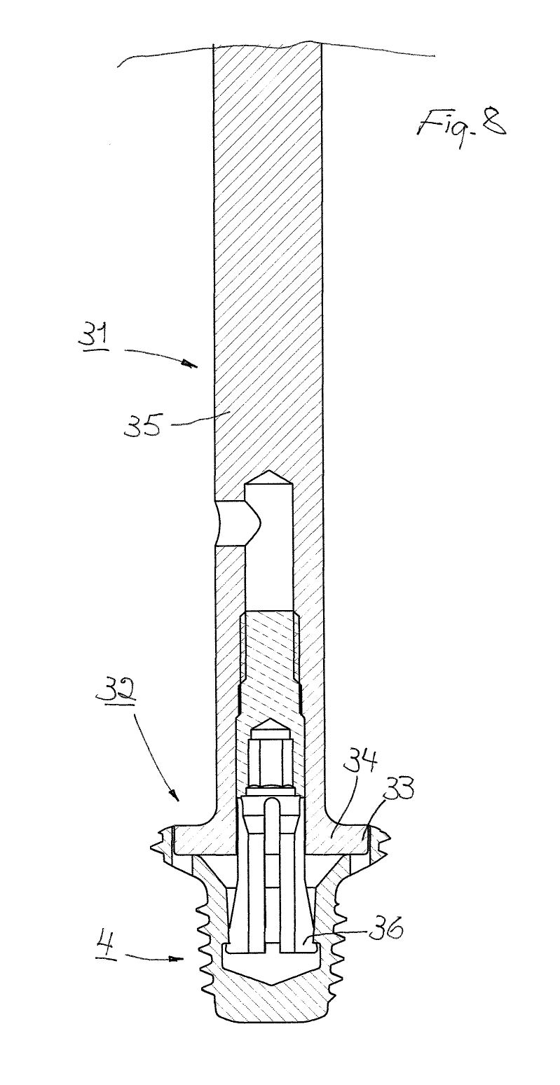

The recess or recesses 27 in the hole 8 in the screw-like part 4 on the inside of the opening 16 thereto, may according to the invention also be used to permit insertion into said recess or recesses of a driving portion of a driving tool for attachment by screwing of the screw-like part to the trapezium T. Such a driving instrument is illustrated in FIG. 7. It is of course possible to configure the screw-like part 4 with one or more other recesses for attachment thereof by screwing by means of a driving instrument and the driving instrument may be designed otherwise than the driving instrument of FIG. 7. The driving portion 32 of the driving instrument 31 however, is in the embodiment of FIG. 7 configured with protrusions 33 which fit into the recesses 27. The recesses 33, the number of which in the illustrated embodiment corresponds to the number of recesses 27, but which also may be less than that, are provided on a plate-like member 34 of the driving portion which is attached to one end of an elongated shaft 35 of the driving instrument 31. The driving portion 32 of the driving instrument 31 may as illustrated also comprise snap-in portions 36 which snap-in into the extended part of the hole 8 close to the bottom 10 of said hole and which retain the driving instrument in operating position for attachment by screwing of the screw-like part 4. The free end of the shaft 35, opposite to the shaft end with the driving portion 32, may be configured with grip portions 37 for cooperation with a rotary movements generating tool, e.g. a drilling machine, or be configured with a handle or grip portions for cooperation with a handle for manual rotation of the driving instrument. By rotating the driving instrument 31 after insertion of the driving portion 32 thereof into the opening 18 to the hole 8 and after bringing the protrusions 33 into engagement with the recesses 27 (see FIG. 8), the screw-like part 4 can be attached to the trapezium T by screwing.

Alternatively, if the size of the screw-like part permits it, the bottom 10 of the hole 8 may also be configured as or with a groove (see FIG. 3), e.g. a hexagon groove, for attachment by screwing of the screw-like part by means of a driving instrument in the shape of e.g. a hexagon wrench. The groove in the bottom 10 of the hole 8 may of course also be shaped otherwise and so may the driving instrument.

A general description of how the various members 4, 6 and 5, 7 respectively, of the prosthesis members of the thumb prosthesis are attached to the trapezium T and to the first metacarpal bone respectively, is found e.g. in the publication SE 533 438 C2 mentioned above and is not described in detail here.

The first and second screw-like parts 4, 5 respectively, may consist of at least one material, and the articulating socket and ball elements 6, 7 respectively, may consist of at least one other material. Thus, each first and second screw-like part 4, 5 respectively, may comprise a core of metallic material with an outer layer of a material which is dissolvable when the first and second screw-like parts 4, 5 respectively, are implanted and which permits secondary fixation of the screw-like parts.

Said core may e.g. consist of a titanium alloy and the dissolvable outer layer may consist of or include calciumphosphate in order to facilitate “growth” thereof to the bone to which the screw-like parts are attached by screwing.

The articulating ball element 7 may as the articulating socket element 6 consist of a CoCrMo-alloy.

Except that the thumb prosthesis is simple and easy to attach, surgery can be kept to a minimum because you just have to cut off smaller parts of the respective bone in order to attach the screw-like parts 4, 5.

The invention is not limited to the embodiment and the alternatives thereto described above, but may vary within the scope of the subsequent claims. Thus, the thumb prosthesis and particularly the prosthesis member for the first metacarpal bone may be given any other shape than the one described above, provided that the prosthesis member comprises a screw-like part and an articulating ball element. The prosthesis member for the first metacarpal bone may e.g. be configured in one piece instead of comprising a separate screw-like part and a separate articulating ball element, as illustrated.

1. Prosthesis member of a joint prosthesis (1) for a thumb joint, wherein the prosthesis member (2) comprises a screw-like part (4) which is adapted for being attached by screwing into the trapezium (T) at the thumb joint and an articulating socket element (6), and wherein the articulating socket element (6) has a mounting pin (9) which is insert-able into a hole (8) in the screw-like part (4) for attaching the articulating socket element thereto, wherein the screw-like part (4) of the prosthesis member (2) is configured with a first fixing portion (15), defining an opening (16) into the hole (8) as well as portions (8a) of the hole for the mounting pin (9) of the articulating socket element (6) close to the opening and provided with a first external thread (17) which is configured to permit attachment by screwing and fastening of said prosthesis member to the hard outer part (T1) of the trapezium (T), and a second fixing portion (18) having a less diameter than the diameter of the first fixing portion (15), defining a bottom (10) to the hole (8) as well as portions (8b) of the hole for the mounting pin (9) of the articulating socket element (6) close to the bottom and provided with a second external thread (19) which is configured to permit attachment by screwing and fastening of the said prosthesis member (2) to the soft inner part (T2) of the trapezium (T); and that said first fixing portion (15) is provided with a surface (20), said surface connecting the threads (17, 19) on said fixing portions (15, 18) to each other and converging substantially arcuate towards the second fixing portion.

2. Prosthesis member according to claim 1, wherein the first fixing portion (15) is configured substantially as a screw head-like portion with through holes (21) which are configured to permit ingrowth of bone tissue therein, with a substantially saddle-shaped side (22) which defines the opening (16) to the hole (8) for the mounting pin (9) of the articulating socket element (6), with a threaded lateral surface (23) and a side defining the surface (20) interconnecting the threads (17, 19) of the fixing portions (15, 18), and that the second fixing portion (18) is configured substantially as a screw shaft-like portion.

3. Prosthesis member according to claim 1, wherein said first and second fixing portions (15, 18) have externally a conical shape.

4. Prosthesis member according to claim 1, wherein said first and second threads (17, 19) on said first and second fixing portions (15, 18) have the same pitch.

5. Prosthesis member according to claim 1, wherein said first and second threads (17, 19) on said first and second fixing portions (15, 18) are conical.

6. Prosthesis member according to claim 1, wherein said first and second threads (17, 19) on said first and second fixing portions (15, 18) are straight.

7. Prosthesis member according to claim 1, wherein the external threads (17, 19) of the first and second fixing portions (15, 18) are self-tapping.

8. Prosthesis member according to claim 1, wherein the hole (8) in the screw-like part (4) and the mounting pin (9) of the articulating socket element (6) are configured with complementary portions for fastening of the articulating socket element in the screw-like part.

9. Prosthesis member according to claim 8, wherein the hole (8) in the screw-like part (4) comprises, at the bottom (10), a snap-in attachment (24) for the mounting pin (9) of the articulating socket element (6) and that the mounting pin (9) of the articulating socket element (6) is configured with snap-in portions (25) which engage the snap-in attachment (24) in the hole (8) in the screw-like part (4).

10. Prosthesis member according to claim 1, wherein the hole (8) in the screw-like part (4) and a portion (28) defining the socket (11) of the articulating socket element (6) are configured with complementary portions which prevent rotation of the articulating socket element relative to the screw-like part.

11. Prosthesis member according to claim 10, wherein the hole (8) in the screw-like part (4) is on the inside of the opening (16) to said hole configured with at least one recess (27) and the articulating socket element (6) is externally on the portion (28) defining the socket (11) configured with at least one protrusion (29) which engage the recess in the hole in the screw-like part to prevent rotation of the articulating socket element relative to the screw-like part.

12. Prosthesis member according to claim 1, wherein the hole (8) in the screw-like part (4) and the mounting pin (9) of the articulating socket element (6) are configured such that said screw-like part and said articulating socket element define a press fit which permits bringing the articulating socket element and the screw-like part to attach to each other by pressing them together.

13. Prosthesis member according to claim 1, wherein the hole (8) in the screw-like part (4) and the mounting pin (9) of the articulating socket element (6) are conically shaped.

14. Prosthesis member according to claim 1, wherein the articulating socket element (6), except for the mounting pin (9), comprises a portion (28) defining the socket (11), said portion being configured with a collar (30).

15. Prosthesis member according to claim 1, wherein the opening (16) to the hole (8) in the first fixing portion (15) of the screw-like part (4) is provided with at least one recess (27) which is configured to permit insertion into said recess of a driving portion (32) of a driving instrument (31) for attachment by screwing of the screw-like part to the trapezium (T).

16. Prosthesis member according to claim 1, wherein the bottom (10) of the hole (8) in the second fixing portion (18) of the screw-like part (4) is configured as or with a groove to permit insertion into said groove of a driving portion of a driving instrument (31) for attachment by screwing of the screw-like part to the trapezium (T).

17. Joint prosthesis for a thumb joint, comprising a prosthesis member according to claim 1, and a prosthesis member (3) with a screw-like part (5) which is configured for attachment by screwing to the first metacarpal bone at the thumb joint and with an articulating ball element (7).
